(19) United States
(12) Patent Application Publication
(10) Pub. No.: US 2026/0007790 A1
(43) Pub. Date: Jan. 8, 2026

(54) PERITONEAL DIALYSIS DISINFECTION DEVICE

(71) Applicants: VANTIVE US HEALTHCARE LLC, Deerfield, IL (US); VANTIVE HEALTH GMBH, Glattpark (Opfikon) (CH)

(72) Inventors: Aporva Raj Makam RAVINDRANATH, Bangalore (IN); Gagan MAHADEVAIAH, Bangalore (IN)

(21) Appl. No.: 19/137,231

(22) PCT Filed: Dec. 6, 2023
(86) PCT No.: PCT/US2023/082666

§ 371 Date: Jun. 9, 2025

(30) Foreign Application Priority Data
Dec. 7, 2022 (IN) 202241070547

(51) Int. Cl. A61L 2/10; A61L 2/00; A61L 2/24; A61M 1/28

(52) U.S. Cl. CPC A61L 2/10; A61L 2/0047; A61L 2/24; A61L 2202/11; A61L 2202/14; A61L 2202/16; A61L 2202/24; A61M 1/28

(57) ABSTRACT

A peritoneal dialysis (“PD”) disinfection device includes a top made of a material configured to let light pass through the material, a bottom, a printed circuit board (“PCB”) secured between the top and the bottom, a plurality of ultraviolet light emitting diodes (“UV-LEDs”) secured to the PCB, and a power source operable to supply power to the plurality of UV-LEDs. At least one of the top, the bottom, and the PCB includes a feature that enables a patient to locate and orient the PD disinfection device so that UV light from the plurality of UV-LEDs is directed towards a connection end of the patient's catheter or a catheter/patient exit site.

### TECHNICAL FIELD

The present disclosure relates generally to medical fluid treatments, and in particular to dialysis fluid treatments.

### BACKGROUND

Due to various causes, a person's renal system can fail. Renal failure produces several physiological derangements. When renal failure occurs, it is no longer possible to balance water and minerals or to excrete daily metabolic load. Toxic end products of metabolism, such as, urea, creatinine, uric acid and others, may accumulate in a patient's blood and tissue.

Reduced kidney function and, above all, kidney failure is treated with dialysis. Dialysis removes waste, toxins, and excess water from the body that normal functioning kidneys would otherwise remove. Dialysis treatment for replacement of kidney functions is critical to many people because the treatment is lifesaving.

One type of kidney failure therapy is Hemodialysis (“HD”), which in general uses diffusion to remove waste products from a patient's blood. A diffusive gradient occurs across the semi-permeable dialyzer between the blood and an electrolyte solution called dialysate or dialysis fluid to cause diffusion.

Hemofiltration (“HF”) is an alternative renal replacement therapy that relies on a convective transport of toxins from the patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi- or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as an in-center patient does, who has built-up two or three days' worth of toxins prior to a treatment. In certain areas, the closest dialysis center can be many miles from the patient's home, causing door-to-door treatment time to consume a large portion of the day. Treatments in centers close to the patient's home may also consume a large portion of the patient's day. HHD can take place overnight or during the day while the patient relaxes, works, or is otherwise productive.

Another type of kidney failure therapy is peritoneal dialysis (“PD”), which infuses a dialysis solution, also called dialysis fluid, into a patient's peritoneal chamber via a catheter. The dialysis fluid is in contact with the peritoneal membrane in the patient's peritoneal chamber. Waste, toxins, and excess water pass from the patient's bloodstream, through the capillaries in the peritoneal membrane, and into the dialysis fluid due to diffusion and osmosis, i.e., an osmotic gradient occurs across the membrane. An osmotic agent in the PD fluid provides the osmotic gradient. Used or spent dialysis fluid is drained from the patient, removing waste, toxins, and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis, and continuous flow peritoneal dialysis (“CFPD”). CAPD is a manual dialysis treatment. Here, the patient manually connects an implanted catheter to a drain to allow used or spent dialysis fluid to drain from the peritoneal chamber. The patient then switches fluid communication so that the patient catheter communicates with a bag of fresh dialysis fluid to infuse the fresh dialysis fluid through the catheter and into the patient. The patient disconnects the catheter from the fresh dialysis fluid bag and allows the dialysis fluid to dwell within the peritoneal chamber, wherein the transfer of waste, toxins, and excess water takes place. After a dwell period, the patient repeats the manual dialysis procedure, for example, four times per day. Manual peritoneal dialysis requires a significant amount of time and effort from the patient, leaving ample room for improvement.

APD is similar to CAPD in that the dialysis treatment includes drain, fill, and dwell cycles. PD machines, however, perform the cycles automatically, typically while the patient sleeps. PD machines free patients from having to manually perform the treatment cycles and from having to transport supplies during the day. PD machines connect fluidly to an implanted catheter, to a source or bag of fresh dialysis fluid and to a fluid drain. PD machines pump fresh dialysis fluid from a dialysis fluid source, through the catheter and into the patient's peritoneal chamber. PD machines also allow for the dialysis fluid to dwell within the chamber and for the transfer of waste, toxins, and excess water to take place. The source may include multiple liters of dialysis fluid including several solution bags.

PD machines pump used or spent dialysate from the patient's peritoneal cavity, though the catheter, and to the drain. As with the manual process, several drain, fill, and dwell cycles occur during dialysis. A “last fill” may occur at the end of the PD treatment. The last fill fluid may remain in the peritoneal chamber of the patient until the start of the next treatment, or may be manually emptied at some point during the day.

Peritonitis is a risk to any type of PD treatment, including APD and CAPD. Peritonitis is an inflammation of the patient's peritoneal cavity. Peritonitis is usually due to a bacterial or fungal infection, which may be brought on by the lack of aseptic handling and connections involved with the setup of the PD treatment. Peritonitis may become very painful and thus the prevention of same is desirable.

Any improved way to prevent peritonitis and to enhance the overall aseptic quality of a PD treatment is needed accordingly.

### SUMMARY

The present disclosure sets forth a peritoneal dialysis (“PD”) disinfection device, which is manually adjustable to disinfect both a connection end of the patient's catheter or transfer set and a catheter/patient exit site. The PD disinfection device in one embodiment outwardly directs ultraviolet (“UV”) light, such that the patient may orient the device such that the outwardly directed UV light irradiates the connection end of the patient's catheter or transfer set or the catheter/patient exit site, whichever is desired. The PD disinfection device may be provided with instructions that inform the patient to orient the device towards (i) the connection end of the patient's catheter or transfer set and to disinfect same before connecting to the patient line for treatment, and (ii) the catheter/patient exit site and to disinfect same after treatment.

The PD disinfection device is, in one embodiment, made from three primary components, namely, a see-through or transparent top (of a material configured to let light, e.g., ultraviolet light, pass through the material), a printed circuit board (“PCB”), and a bottom, which is in one embodiment opaque. The top and bottom include mating features so that the top and bottom fit together around the printed circuit board. The mating features of the top and bottom may be configured to snap-fit together or be provided with openings and threaded bores to accept one or more screw or bolt to secure the top and bottom. The weight of the PD disinfection device may be about 48 grams and have an expected shelf life of at least five years.

The see-through or transparent top may have a refractive index of 1.5, wherein both the top and bottom may be made of plastic. The top and bottom are in an embodiment made of biocompatible materials so as to allow the patient to handle the disinfection device during and around a PD treatment, and wherein the materials are compatible for use with disinfectants and associated wipes, sodium hypochloride, ammonium chloride, 60601-1 ethanol, and isopropyl alcohol.

The UV lights are in one embodiment UV light emitting diodes (“LED's”) and are connected, e.g., soldered, to the PCB. In an embodiment, seven UV-LEDs are provided, which may each operate at 5 VDC at 15 mA and have a 130 degree viewing angle. So at a maximum, each UV-LED will require 20 mA current. Each UV-LED may further be configured to emit UV light at a wavelength within the range of 265 nm to 285 nm, which is well-suited for avoiding or destroying pathogens. The disinfection device instructions may further inform the patient to maintain the disinfection device at a distance of ten to twelve centimeters away from the area to be disinfected, e.g., the connection end of the patient's catheter or transfer set and the catheter/patient exit site. The UV light may be blue or purple in color such that the patient can readily identify when the UV light is properly focused on the area to be disinfected.

The PD disinfection device further includes a universal serial bus (“USB”) connector for connecting to a mating USB connector of a power cord for powering the disinfection device. The USB connector is in an embodiment soldered to the PCB, wherein electrical traces run along the PCB from the USB connector to a rechargeable, e.g., 5 VDC, power supply or battery (coin cell), which is likewise soldered to the PCB. The rechargeable power supply or battery may have a 150 mAh capacity, which can run 7.5 hours off of one charge ideally. A light emitting diode (“LED”) may be soldered to the PCB and be electrically connected to the rechargeable power supply or battery. The LED may operate for example on 0.6 VDC light and be configured to light green when the power supply or battery is fully charged and to light red when the power supply or battery is low on charge, e.g., below twenty-five percent.

The PD disinfection device in an embodiment includes a switch, such as a maintained on-off slide type switch, which may also be soldered to the PCB. When the switch is off, no power is suppled from the battery to the UV-LEDs. When the patient moves the switch to the on position, the power supply powers the UV-LEDs, which cast UV light for disinfecting the catheter/patient exit site or a connection end of the patient's catheter or transfer set. In an embodiment, the rechargeable power supply or battery may be recharged regardless of the position of the on-off switch.

The PD disinfection device may further include a timer, such as a NE555 timer circuit IC(2), which is configured to automatically cut power to the UV-LEDs after a certain period of time, e.g., thirty minutes or more, as a safety feature to avoid overexposure to UV light, especially at the catheter/patient exit site.

Additional electrical traces are applied to the PCB to electrically connect (i) the on-off switch between the rechargeable power supply or battery and the timer circuit, and (ii) the timer circuit between the rechargeable power supply or battery and each of the, e.g., seven, UV-LEDs. Thus either the on-off switch or the timer circuit is positioned to cut power to the UV-LEDs. One or more electrical trace is applied to the PCB to electrically connect the red/green power level LED and the rechargeable power supply or battery. In an embodiment, both sides of the PCB are used to maximize PCB space efficiency. For example, the UV-LEDs, the red/green power level LED, and the timer circuit may be provided on a top surface of the PCB, while the USB connector, the rechargeable power supply or battery, and the on-off switch are provided on a bottom surface of the PCB.

It is contemplated to mount the PD disinfection device in a plurality of different ways for operation. In a first embodiment, each of the see-through or transparent top, the PCB, and the bottom are hinged via a hinge to allow an opposing end of the PD disinfection device to open and be placed around the patient's catheter or transfer set. Once the PD disinfection device is placed around the patient's catheter or transfer set, the patient closes the PD disinfection device at the opposing end by closing the hinge. The patient hingedly closes the PD disinfection device about the patient's catheter, such that the UV light points either towards catheter/patient exit site or the connection end of the patient's catheter or transfer set. A snap-fit may be made at the opposing end to hold the PD disinfection device closed about the patient's catheter or transfer set until it is desired to remove the disinfection device.

In a second embodiment, the see-through or transparent top, the PCB, and the bottom of the device are each provided with a slot, wherein the slots align when the disinfection device is assembled. Here, the patient inserts the PD disinfection device over the patient's catheter via the slot, such that the UV light points either towards the catheter/patient exit site or the connection end of the patient's catheter or transfer set. One or more snap features may be provided with the slot at one or more of the see-through or transparent top and/or the bottom, so that the patient's catheter is discouraged from slipping out from the device via the slot during use.

In a third embodiment, which may be provided alternatively or additionally with the slot, the PD disinfection device, e.g., the bottom of the PD disinfection device, is provided or formed with a looping member that defines an opening through which a chain or pendant worn by the patient is inserted. Here, the PD disinfection device hangs from the chain or pendant worn around the neck of the patient in a manner such that the PD disinfection device resides close to the catheter/patient exit for emitting UV light onto same.

In a fourth embodiment, which may be provided alternatively or additionally with the slot and/or the looping member, a flexible mechanical link, e.g., a chain-like link, is provided. One end of the flexible mechanical link is configured to connect to the patient's belt or pant loop. The other end of the flexible mechanical link is configured to connect to a mating feature provided on the PD disinfection device, e.g., on the bottom of the PD disinfection device. The flexible mechanical link is configured to be articulated (e.g., anywhere between zero and 360 degrees) into a desired and held position such that the UV light from the PD disinfection device is emitted onto a desired area, e.g., onto the catheter/patient exit.

In light of the disclosure set forth herein, and without limiting the disclosure in any way, in a first aspect, which may be combined with any other aspect or portion thereof, a peritoneal dialysis (“PD”) disinfection device includes a top made of a material configured to let light pass through the material, a bottom, a printed circuit board (“PCB”) secured between the top and the bottom, a plurality of ultraviolet light emitting diodes (“UV-LEDs”) secured to the PCB, and a power source operable to supply power to the plurality of UV-LEDs. At least one of the top, the bottom, or the PCB includes at least one feature that enables a patient to locate and orient the PD disinfection device so that UV light from the plurality of UV-LEDs is directed towards a connection end of the patient's catheter or a catheter/patient exit site.

In a second aspect of the present disclosure, which may be combined with any other aspect or portion thereof, the top is made of a transparent material.

In a third aspect of the present disclosure, which may be combined with any other aspect or portion thereof, the top and the bottom include mating features for securing the top to the bottom, the mating features including features for bolting the top and the bottom together or snap-fitting the top and the bottom together.

In a fourth aspect of the present disclosure, which may be combined with any other aspect or portion thereof, the bottom is opaque.

In a fifth aspect of the present disclosure, which may be combined with any other aspect or portion thereof, the power supply includes a rechargeable battery.

In a sixth aspect of the present disclosure, which may be combined with any other aspect or portion thereof, the PD disinfection device further includes a connector for electrically connecting to a power cord. The power supply is configured to be charged via the power cord.

In a seventh aspect of the present disclosure, which may be combined with any other aspect or portion thereof, the connector is at least one of (i) a universal serial bus (“USB”) connector or (ii) secured to the PCB.

In an eighth aspect of the present disclosure, which may be combined with any other aspect or portion thereof, the PD disinfection device further includes a switch positioned and arranged to enable power to be selectively supplied from the power source to the plurality of UV-LEDs.

In a ninth aspect of the present disclosure, which may be combined with any other aspect or portion thereof, the switch is at least one of (i) sized to extend through an opening defined by the top or the bottom, or (ii) secured to the PCB.

In a tenth aspect of the present disclosure, which may be combined with any other aspect or portion thereof, the PD disinfection device further includes a timer circuit configured to remove power from the plurality of UV-LEDs after a certain period of time.

In an eleventh aspect of the present disclosure, which may be combined with any other aspect or portion thereof, at least one of (i) the certain period of time is thirty minutes or more, or (ii) the timer circuit is secured to the PCB.

In a twelfth aspect of the present disclosure, which may be combined with any other aspect or portion thereof, the at least one feature includes a combined slot formed in the top, the PCB, and the bottom.

In a thirteenth aspect of the present disclosure, which may be combined with any other aspect or portion thereof, the at least one feature includes a looping member defining an opening through which a chain or pendant worn by the patient is inserted.

In a fourteenth aspect of the present disclosure, which may be combined with any other aspect or portion thereof, the at least one feature includes a flexible mechanical link configured to connect to one of the top or bottom and the patient's belt or pant loop.

In a fifteenth aspect of the present disclosure, which may be combined with any other aspect or portion thereof, a PD disinfection device includes a top made of a material configured to let light pass through the material, a bottom, a printed circuit board (“PCB”) secured between the top and the bottom, a plurality of ultraviolet light emitting diodes (“UV-LEDs”) secured to the PCB, and a combined slot formed in the top, the PCB, and the bottom that enables a patient to locate and orient the PD disinfection device about the patient's catheter so that UV light from the plurality of UV-LEDs is directed towards a connection end of the patient's catheter or a catheter/patient exit site.

In a sixteenth aspect of the present disclosure, which may be combined with any other aspect or portion thereof, the combined slot includes at least one snap feature configured to discourage the patient's catheter from slipping out from the combined slot during use.

In a seventeenth aspect of the present disclosure, which may be combined with any other aspect or portion thereof, the PD disinfection device further includes a looping member defining an opening through which a chain or pendant worn by the patient is inserted.

In an eighteenth aspect of the present disclosure, which may be combined with any other aspect or portion thereof, the looping member is provided or formed with the bottom.

In a nineteenth aspect of the present disclosure, which may be combined with any other aspect or portion thereof, the PD disinfection device further includes a flexible mechanical link configured to connect to one of the top or bottom and the patient's belt or pant loop.

In a twentieth aspect of the present disclosure, which may be combined with any other aspect or portion thereof, any of the features, functionality and alternatives described in connection with any one or more of FIGS. 1 to 8 may be combined with any of the features, functionality and alternatives described in connection with any other of FIGS. 1 to 8 and/or any of the aspects listed herein.

It is accordingly an advantage of the present disclosure to provide an improved PD disinfection device.

It is another advantage of the present disclosure to provide a PD disinfection device that is able to disinfect a catheter/patient exit site.

It is a further advantage of the present disclosure to provide a PD disinfection device that is adjustable so as to be able to disinfect a catheter/patient exit site in addition to disinfecting a connection end of the patient's catheter or transfer set.

It is yet another advantage of the present disclosure to provide a PD disinfection device that is safe for the patient and safe for a PD treatment.

It is yet a further advantage of the present disclosure to provide a PD disinfection device that is cost efficient.

It is still another advantage of the present disclosure to provide a PD disinfection device that is lightweight and easy to maneuver by the patient.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a perspective view of one embodiment for an assembled peritoneal dialysis (“PD”) disinfection device of the present disclosure.

FIG. 2 is a first exploded view of one embodiment for the PD disinfection device of the present disclosure.

FIG. 3 is a second exploded view of one embodiment for the PD disinfection device of the present disclosure.

FIG. 4 is a top view of one embodiment for the PD disinfection device of the present disclosure, illustrating one way to recharge the device.

FIG. 5 is a side elevation view of one embodiment for the PD disinfection device of the present disclosure oriented so as to disinfect a connection end of the patient's catheter or transfer set.

FIG. 6 is a side elevation view of one embodiment for the PD disinfection device of the present disclosure oriented so as to disinfect a catheter/patient exit site.

FIG. 7 shows various views of an alternative embodiment for the PD disinfection device of the present disclosure, which is worn around the neck of the patient.

FIG. 8 shows a side elevation view of another alternative embodiment for a PD disinfection device of the present disclosure, which is attached to a belt worn by the patient.

### DETAILED DESCRIPTION

### System Overview

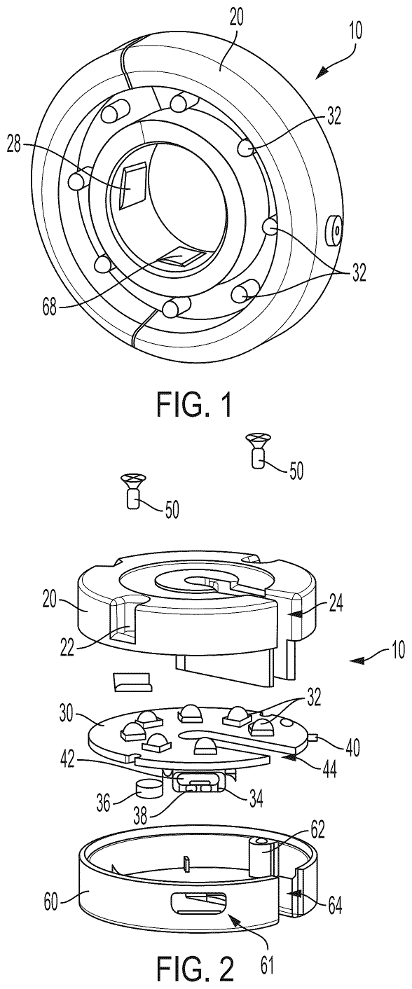

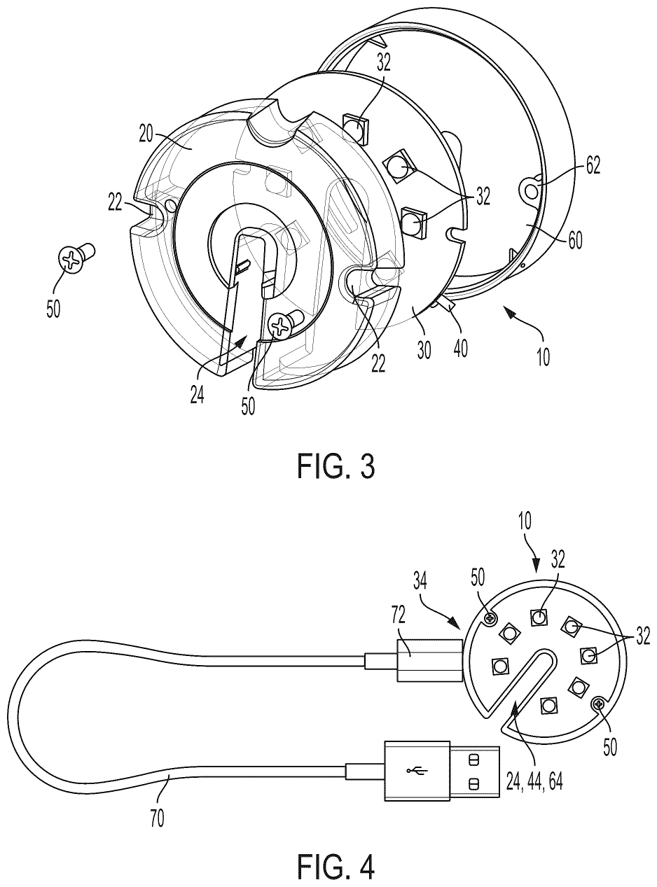

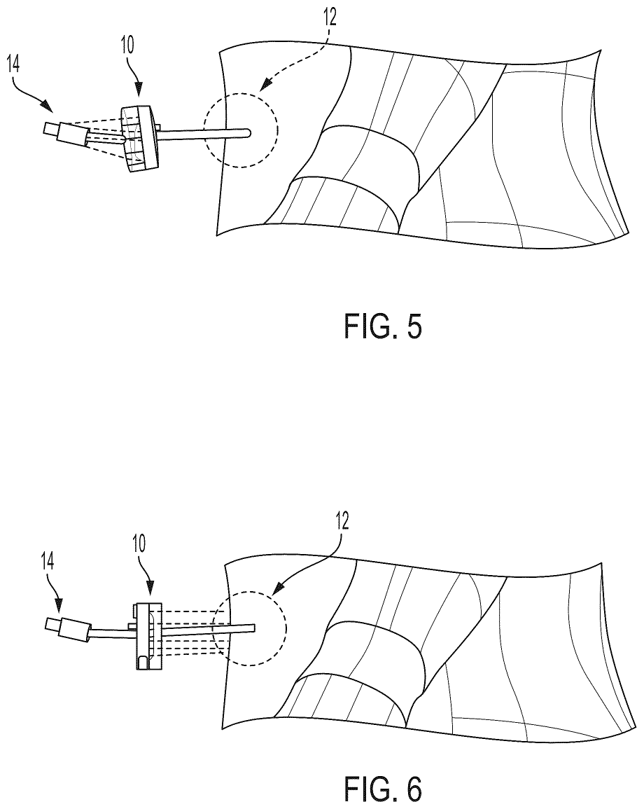

Referring now to the drawings and in particular to FIGS. 1 to 3, a peritoneal dialysis (“PD”) disinfection device 10 is illustrated. PD disinfection device 10 is manually adjustable to disinfect both a catheter/patient exit site 12 (FIGS. 6 to 8) and a connection end of the patient's catheter or transfer set 14 (FIGS. 5 to 7). PD disinfection device 10 in one embodiment outwardly directs ultraviolet (“UV”) light, such that the patient may orient the device such that the outwardly directed UV light irradiates the connection end of the patient's catheter or transfer set 14 or catheter/patient exit site 12, whichever is desired. PD disinfection device 10 may be provided with instructions that inform the patient to orient device 10 towards (i) the connection end of the patient's catheter or transfer set 14 and to disinfect the same before connecting to the patient line for treatment, and (ii) catheter/patient exit site 12 and to disinfect the same after treatment.

PD disinfection device 10 is in one embodiment made from three primary components, namely, a see-through or transparent top 20 (made of a material configured to let light, e.g., ultraviolet light, pass through the material), a printed circuit board (“PCB”) 30 and a bottom 60, which is in one embodiment opaque. Top 20 and bottom 60 include mating, e.g., molded, features 22, 62 so that top 20 and bottom 60 fit together around the printed circuit board. Mating features 22, 62 of top 20 and bottom 60 may be configured to snap-fit together or be provided with openings and threaded bores to accept one or more screw or bolt 50 to secure top 20 and bottom 60. The weight of PD disinfection device 10 may be about forty-eight grams and have an expected shelf life of at least five years.

See-through or transparent top 20 may have a refractive index of 1.5, wherein both the top and bottom 60 may be made of one or more plastic, e.g., Acrylonitrile butadiene styrene (“ABS”), polyvinylchloride (“PVC”) or a non-PVC material, such as polyethylene (“PE”), polyurethane (“PU”), polycarbonate (“PC”), ABS combined with PC, and/or polyetheretherketone (“PEEK”). Bottom 60 may be made alternatively or additionally of metal, e.g., stainless steel, steel, and/or aluminum. Top 20 and bottom 60 are in an embodiment made of biocompatible materials. In some instances, the materials are compatible for use with disinfectants and associated wipes, sodium hypochloride, ammonium chloride, 60601-1 ethanol, and isopropyl alcohol to allow the patient to handle the disinfection device 10 during and around a PD treatment.

The UV lights are in one embodiment UV light emitting diodes (“LEDs”) 32 and are connected, e.g., soldered, to PCB 30. In an embodiment, seven UV-LEDs 32 are provided, which may each operate at 5 VDC with rated current of 15 mA and have a 130 degree viewing angle. So at a maximum, each UV-LED 32 requires 20 mA current. Each UV-LED 32 may further be configured to emit UV light at a wavelength within the range of 265 nm and 285 nm, which is well-suited for avoiding or destroying pathogens. PD disinfection device 10 instructions may further inform the patient to maintain the disinfection device at a distance of ten to twelve centimeters away from the area to be disinfected, e.g., the connection end of the patient's catheter or transfer set 14 and catheter/patient exit site 12. The UV light may be blue or purple in color such that the patient can readily identify when the UV light is properly focused on the area to be disinfected.

FIGS. 2 and 4 illustrates that PD disinfection device 10 further includes a universal serial bus (“USB”) connector 34 for connecting to a mating USB connector 72 of a power cord 70 for powering disinfection device 10. FIG. 2 illustrates that bottom 60 defines a hole or opening 61 for enabling USB connector 72 to enter device 10 and connect to USB connector 34. USB connector 34 is in an embodiment soldered to PCB 30, wherein electrical, e.g. copper, traces (not illustrated) run along PCB 30 from USB connector 34 to a rechargeable, e.g., 5 VDC, power supply or battery 36 (e.g., coin cell battery), which is inserted replaceably into a power supply holder (not illustrated) that is likewise soldered to PCB 30. Rechargeable power supply or battery 36 may have a 150 mAh capacity, which can run 7.5 hours off of one charge ideally. An LED 38 may be soldered to PCB 30 and be electrically connected to rechargeable power supply or battery 36. LED 38 may operate for example on 0.6 VDC light and be configured to light green when power supply or battery 36 is fully charged and to light red when power supply or battery 36 is low, e.g., below twenty-five percent, on charge.

PD disinfection device 10 in an embodiment includes a switch 40, such as a maintained on-off slide type switch, which may also be soldered to PCB 30 and extend through an opening provided by bottom 60. When switch 40 is off, no power is suppled from power supply or battery 36 to UV-LEDs 32. When the patient moves switch 40 to the on position, power supply or battery 36 powers UV-LEDs 32, which cast UV light for disinfecting catheter/patient exit site 12 or the connection end of the patient's catheter or transfer set 14. In an embodiment, rechargeable power supply or battery 36 may be recharged regardless of the position of on-off switch 40.

PD disinfection device 10 may further include a timer 42, such as a NE555 timer circuit IC(2), which is configured to automatically cut power to the UV-LED's 32 after a certain period of time, e.g., thirty minutes, as a safety feature to avoid overexposure to UV light, especially at catheter/patient exit site 12.

Additional electrical traces (not illustrated) are applied to PCB 30 to electrically connect (i) on-off switch 40 between rechargeable power supply or battery 36 and timer circuit 42, and (ii) timer circuit 42 between rechargeable power supply or battery 36 and each of the, e.g., seven, UV-LEDs 32. Thus either on-off switch 40 or timer circuit 42 is positioned to cut power to UV-LEDs 32. One or more electrical trace (not illustrated) is applied to PCB 30 to electrically connect green/red power level LED 38 and rechargeable power supply or battery 36. In an embodiment, both sides of PCB 30 are used to maximize PCB space efficiency. For example, UV-LED's 32, green/red power level LED 38 and timer circuit 42 may be provided on, e.g., soldered to, a top surface of PCB 30, while USB connector 34, rechargeable power supply or battery 36, and on-off switch 40 are provided on, e.g., soldered to, a bottom surface of PCB 30.

In some embodiments, switch 40 and timer circuit 42 may be located in series with UV-LEDs 32, which are electrically in parallel with each other. Thus when switch 40 is closed and timer circuit 42 has not yet timed out, each of UV-LEDs 32 is powered simultaneously. Further, in some instances USB connector 34 is electrically connected between the positive and negative terminals of power supply or battery 36 for recharging the power supply or battery when power cord 70 is plugged into USB connector 34. A voltage meter may be located between the positive and negative terminals of power supply or battery 36 for detecting a stored voltage level for the supply or battery. Voltage meter has two switches, one for lighting green/red LED 38 green when power supply or battery 36 is fully charged, the other for lighting for lighting green/red LED 38 red when the power supply or battery is low on charge, e.g., below twenty-five percent.

It is contemplated to mount PD disinfection device 10 in a plurality of different ways for operation. FIG. 1 illustrates a first embodiment in which each of see-through or transparent top 20, PCB 30, and bottom 60 are hinged via a hinge 28, 68 to allow an opposing end of PD disinfection device 10 to open and be placed around the patient's catheter or transfer set 14. Once PD disinfection device 10 is placed around the patient's catheter or transfer set 14, the patient closes the PD disinfection device at the opposing end by closing hinge 28, 68. The patient hingedly closes PD disinfection device 10 about the patient's catheter 14, such that the UV light points either towards catheter/patient exit site 12 or the connection end of the patient's catheter or transfer set 14. A snap-fit may be made at the opposing end to hold PD disinfection device 10 closed about the patient's catheter or transfer set 14 until it is desired to remove the disinfection device.

FIGS. 2 to 6 illustrate a second embodiment in which see-through or transparent top 20, PCB 30, and bottom 60 of the device are each provided with a slot 24, 44, 64, wherein slots 24, 44, 64 align when PD disinfection device 10 is assembled. Here, the patient inserts PD disinfection device 10 over the patient's catheter 14 via combined slot 24, 44, 64, such that the UV light points either towards catheter/patient exit site 12 or the connection end of the patient's catheter or transfer set 14. FIG. 4 best illustrates that one or more snap features 26 may be provided with at slot 24 via one or more of see-through or transparent top 20 and/or bottom 60, so that the patient's catheter 14 is discouraged from slipping out from the device via combined slot 24, 44, 64 during use.

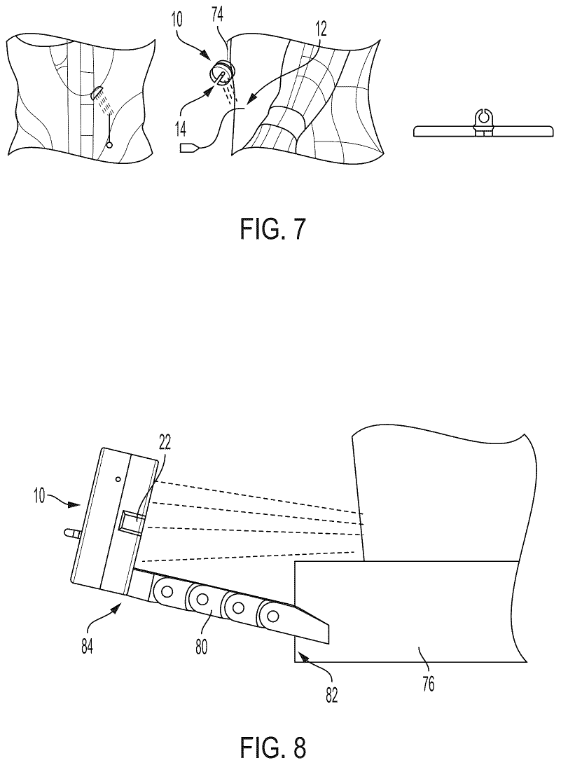

FIG. 7 illustrates a third embodiment, which may be provided alternatively or additionally with the slot 24, 44, 64. Here, PD disinfection device 10, e.g., bottom 60 of the PD disinfection device, is provided or formed with a looping member 66 that defines an opening through which a chain or pendant 74 worn by the patient is inserted. PD disinfection device 10 hangs from chain or pendant 74 worn around the neck of the patient in a manner such that the PD disinfection device resides close to the catheter/patient exit site 12 for emitting UV light onto same. Combined slot 24, 44, 64 of device 10 may additionally and simultaneously be inserted over the patient's catheter or transfer set 14.

FIG. 8 illustrates a fourth embodiment, which may be provided alternatively or additionally with the slot 24, 44, 64 and/or the looping member 66, wherein a flexible mechanical link 80, e.g., a chain-like link is provided. A first end 82 of flexible mechanical link 80 is configured to connect to the patient's belt 76 or pant loop. The other, second end 84 of flexible mechanical link 80 is configured to connect to a mating feature (not illustrated) provided on PD disinfection device 10, e.g., on top 20 or bottom 60 of the PD disinfection device. Flexible mechanical link 80 is configured to be articulated (e.g., anywhere between zero and 360 degrees) into a desired and held position such that the UV light from PD disinfection device 10 is emitted onto a desired area, e.g., onto catheter/patient exit 12.

It should be understood that various changes and modifications to the presently preferred embodiments described herein will be apparent to those skilled in the art. It is therefore intended that such changes and modifications be covered by the appended claims.

1: A peritoneal dialysis (“PD”) disinfection device comprising:
a top made of a material configured to let light pass through the material;
a bottom;
a printed circuit board (“PCB”) secured between the top and the bottom;
a plurality of ultraviolet light emitting diodes (“UV-LEDs”) secured to the PCB; and
a power source operable to supply power to the plurality of UV-LEDs,
wherein at least one of the top, the bottom, or the PCB includes at least one feature that enables a patient to locate and orient the PD disinfection device so that UV light from the plurality of UV-LEDs is directed towards a connection end of a catheter of the patient or a catheter/patient exit site, and
wherein the at least one feature includes a combined slot formed in the top, the PCB, and the bottom.

2: The PD disinfection device of claim 1, wherein the top is made of a transparent material.

3: The PD disinfection device of claim 1, wherein the top and the bottom include mating features for securing the top to the bottom, the mating features including features for bolting the top and the bottom together or snap-fitting the top and the bottom together.

4: The PD disinfection device of claim 1, wherein the bottom is opaque.

5: The PD disinfection device of claim 1, wherein the power supply includes a rechargeable battery.

6: The PD disinfection device of claim 1, which includes a connector for electrically connecting to a power cord, and w herein the power supply is configured to be charged via the power cord.

7: The PD disinfection device of claim 6, wherein the connector is at least one of (i) a universal serial bus (‘“USB”) connector or (ii) secured to the PCB.

8: The PD disinfection device of claim 1, which includes a switch positioned and arranged to enable power to be selectively supplied from the power source to the plurality of UV-LEDs.

9: The PD disinfection device of claim 8, wherein the switch is at least one of (i) sized to extend through an opening defined by the top or the bottom, or (ii) secured to the PCB.

10: The PD disinfection device of claim 1, which includes a timer circuit configured to remove power from the plurality of UV-LEDs after a certain period of time.

11: The PD disinfection device of claim 10, wherein at least one of (i) the certain period of time is thirty minutes or more, or (ii) the timer circuit is secured to the PCB.

12: (canceled)

13: The PD disinfection device of claim 1, wherein the at least one feature includes a looping member defining an opening through which a chain or pendant worn by the patient is inserted.

14: The PD disinfection device of claim 1, wherein the at least one feature includes a flexible mechanical link configured to connect to one of the top or the bottom and a belt or a pant loop.

15: A peritoneal dialysis (“PD”) disinfection device comprising:
a top made of a material configured to let light pass through the material;
a bottom;
a printed circuit board (“PCB”) secured between the top and the bottom;
a plurality of ultraviolet light emitting diodes (“UV-LEDs”) secured to the PCB; and
a combined slot formed in the top, the PCB, and the bottom that enables a patient to locate and orient the PD disinfection device about a catheter of the patient so that UV light from the plurality of UV-LEDs is directed towards a connection end of the catheter of the patient or a catheter/patient exit site.

16: The PD disinfection device of claim 15, wherein the combined slot includes at least one snap feature configured to discourage the catheter of the patient from slipping out from the combined slot during use.

17: The PD disinfection device of claim 15, which further includes a looping member defining an opening through which a chain or pendant worn by the patient is inserted.

18: The PD disinfection device of claim 17, wherein the looping member is provided or formed with the bottom.

19: The PD disinfection device of claim 15, further comprising a flexible mechanical link configured to connect to one of the top or the bottom and a belt or a pant loop.
